(19) United States
(12) Patent Application Publication
(10) Pub. No.: US 2013/0178898 A1
(43) Pub. Date: Jul. 11, 2013

(54) TISSUE APPROXIMATION

(71) Applicants: Jeffery Arnett, Gilbert, AZ (US); Dylan M. Hushka, Gilbert, AZ (US)

(72) Inventors: Jeffery Arnett, Gilbert, AZ (US); Dylan M. Hushka, Gilbert, AZ (US)

(73) Assignee: IMDS Corporation, Logan, UT (US)

(21) Appl. No.: 13/543,514

(22) Filed: Jul. 6, 2012

Related U.S. Application Data
(60) Provisional application No. 61/504,991, filed on Jul. 6, 2011.

(51) Int. Cl. A61B 17/04

(52) U.S. Cl. CPC A61B 17/0487; A61B 17/0401
USPC 606/224

(57) ABSTRACT

Systems and methods for approximating tissue using suture are described. The systems include at least one suture with a locking portion that is integral to the suture. The locking portion may be an expanded portion of the suture body. The locking portion may receive a plurality of snares that work to carry the at least one end of the suture through the locking portion. The systems may also include suture anchors and external locking mechanisms such as a tubular sheath. The systems may be used to approximate two soft tissue portions together, or a soft tissue and a bone portion together.

### CROSS-REFERENCE TO RELATED APPLICATIONS

This application claims the benefit of:

U.S. Provisional Patent Application No. 61/504,991, filed Jul. 6, 2011, entitled TISSUE APPROXIMATION WITH SUTURE, Attorney's docket no. MLI-99 PROV, which is pending.

### BACKGROUND OF THE INVENTION

The present disclosure relates to a tissue approximation system that employs suture and anchors in order to secure tissues together, and to reduce or eliminate the cost and time associated with tying suture knots. Tying suture knots may require a significant amount of time, and may result in bulky surgical knots. Furthermore, the strength of the repair may be limited by the strength of the knot, as the strength of a suture knot may be lower than the tensile strength of the strand.

To overcome these drawbacks, sutures and methods are disclosed that include a passageway for forming a suture loop. In this system, one end of the suture is passed through a passage that may be defined within the body of the suture itself. At least one end of the suture may be pulled through the passage to form a loop that may be tightly closed to bring two tissues into close proximity without the need to tie a surgical knot.

### BRIEF DESCRIPTION OF THE DRAWINGS

Various examples of the present technology will now be discussed with reference to the appended drawings. It is appreciated that these drawings depict only typical examples of the invention and are therefore not to be considered limiting of its scope.

FIG. 1 is a front view of a tissue approximation system, which includes a suture and suture snares;

FIG. 2 is a front view of a suture with a needle passed through the body to form a lock portion;

FIG. 3 is a front view of the system of FIG. 1 after a first suture end has been passed through tissue and through an open loop of a snare, and a second suture end has been passed through tissue and another open loop of a snare;

FIG. 4 is a front view of the system of FIG. 1 after the suture ends and snares have been pulled through the lock portion;

FIG. 5 is a front view of the system of FIG. 1 after the suture snares have been discarded and the suture ends have been pulled tight to approximate the tissue;

FIG. 6 is a front view of a larger version of the system of FIG. 1 that may be used as a tourniquet;

FIG. 7 is a method diagram for approximating tissue using a suture system with a lock portion;

FIG. 8 is a front view of another tissue approximation system, which includes a suture, suture snares, and suture anchor;

FIG. 9 is a front view of the system of FIG. 8 after a first suture end has been passed through tissue and the open loop of a first suture snare, and after a second suture end has been passed through tissue and through the open loop of a second suture snare;

FIG. 10 is a front view of the system of FIG. 8 after the first suture end and corresponding suture snare have been pulled through the lock portion, and after the second suture end and corresponding suture snare have been pulled through the anchor portion and the lock portion;

FIG. 11 is a front view of the system of FIG. 8 after the suture snares have been discarded, the suture anchor has been inserted and deployed in a bone tunnel and the suture ends have been pulled tight to approximate the tissue and the bone;

FIG. 12 is a method diagram for approximating tissue using a suture with a lock portion and a suture anchor component;

FIG. 13 is a front view of another snare formed from a strand that is doubled back on itself;

FIG. 14 is a front view of yet another snare formed from a doubled strand whose ends are tied with a knot;

FIG. 15 is a front view of yet another tissue approximation system with a suture, a tissue piercing component and a sliding bumper;

FIG. 16 is a front view of the system of FIG. 15 after the suture has been passed through a first and second tissue portion;

FIG. 17 is a front view of the system of FIG. 15 after the suture has been pulled to engage the tissue piercing component with tissue;

FIG. 18 is a front view of the system of FIG. 15 after the sliding bumper has been slid along the suture to engage an opposition portion of tissue than the tissue piercing component to approximate the first and second tissue portions;

FIG. 19 is a front view of yet another tissue approximation system with a suture, a needle and an anchor plate;

FIG. 20 is a front view of the system of FIG. 19 after the needle and a first end portion of the suture have been passed through a first and second tissue portion in a first direction, passed back through the first and second tissue portions in a second direction, and has been passed through an aperture in the anchor plate;

FIG. 21 is a front view of yet another tissue approximation system with a suture, a tubular sleeve, a snare with a loop and stalk, and a needle;

FIG. 22 is a front view of the system of FIG. 21 after the suture and needle have been passed through a first and second portion of tissue in a first direction, and passed back through the first and second portions of tissue in a second direction, and through a loop of the snare, and the tubular sleeve has been moved into contact with the first portion of tissue and contains a portion of the stalk;

FIG. 23 is a front view of the system of FIG. 21 after the snare has been passed through the tubular sleeve, pulling the suture and needle through the sleeve;

FIG. 24 is yet another tissue approximation system with a suture with a lock portion and an elongated tubular sleeve;

FIG. 25 is a front view of a suture containing a lock portion;

FIG. 26 is a front view of the suture and lock portion of FIG. 25 with a tube inserted into the lock portion;

FIG. 27 is a front view of yet another tissue approximation system with a first suture formed in a double loop and a second suture used to create a lock portion;

FIG. 28 is a front view of the tissue approximation system of FIG. 1 with an additional lock tube attached to the free suture ends;

FIG. 29 is a front view of the tissue approximation system of FIG. 28 with the lock tube placed in contact with the lock portion of the suture;

FIG. 30 is a front view of the tissue approximation system of FIG. 28 with the lock tube contacting the lock portion of the suture and secured to the suture via crimping;

FIG. 31 is a front view of yet another tissue approximation system including a suture and a first and second exterior sheath embedded within a first and second tissue;

FIG. 32 is a front view of the system of FIG. 31 after the suture has been pulled to create a bunched section of an exterior sheath within the first tissue;

FIG. 33 is a front view of the system of FIG. 31 with the second exterior sheath placed between two tissues; and

FIG. 34 is a front view of the system of 31 with the second exterior sheath placed between two tissues after the suture has been pulled to create a bunched portion of the second exterior sheath to anchor the system between the two tissues.

### DETAILED DESCRIPTION

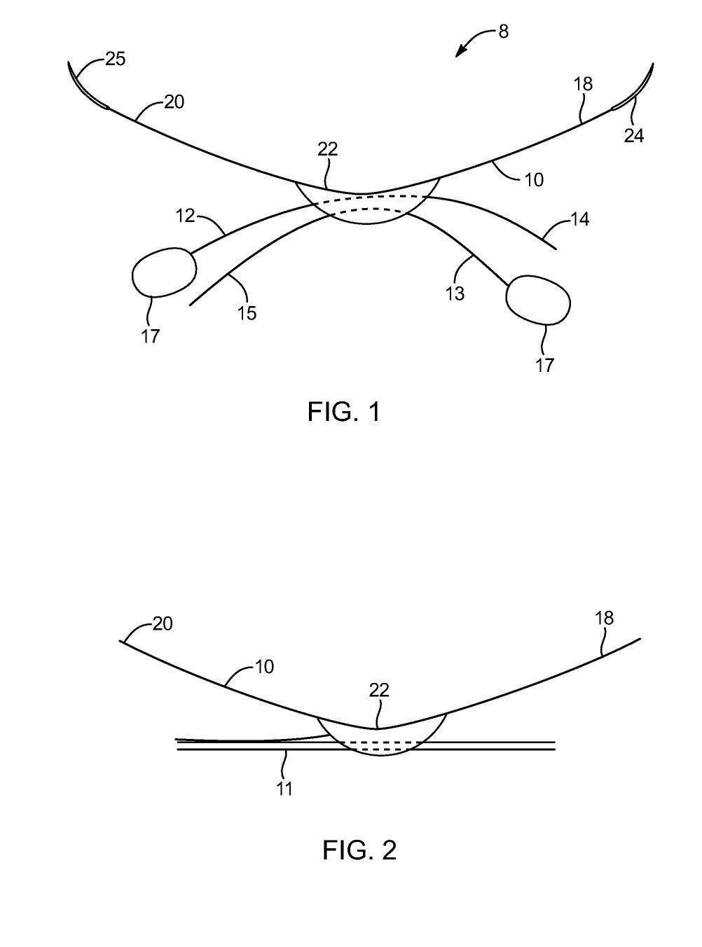

Referring to FIG. 1, a tissue approximation system 8 may include at least one flexible member and at least one suture carrier feature. In the example shown in FIG. 1, the system includes a single flexible strand 10 and two suture carrier features 12, 13. However, any number of flexible members or suture carrier features may be included.

The flexible member 10 may be a suture and the suture carrier features 12, 13 may be snares. The first snare 12 may include a first tail portion 14, which may also be referred as a stalk portion, and a first suture capture portion 16. The second snare 13 may include a second tail portion 15 and a second suture capture portion 17. The suture capture portions 16, 17 may be a flexible, loop-like structure, which may otherwise be polygonal or hook like. Also contemplated, the snare may be a strand that is folded in half such that the bend forms the loop and the ends form the stalk. The first and second capture portions 16, 17 may be integral with the first and second tail portion 14, 15. The snares 12, 13 may be made of a wire-like material, or may otherwise be made of a suture material.

The suture 10 may extend between a first end 18 and a second end 20, and may include a locking portion 22 that lies in between the first end 18 and the second end 20. In the example shown in FIG. 1, the system 8 includes a single locking portion 22, however, multiple locking portions are also contemplated.

The first end 18 of the suture 10 may include a first needle 24 or other tissue-piercing device. The second end 20 of the suture 10 may include a second needle 25 or other tissue-piercing device. The locking portion 22 may also be referred to as a lock portion, trapping portion or a suture pass. The locking portion 22 may be integral to the suture 10, and may include a portion of the suture differentiated by a distinct configuration or weave. The locking portion 22 may otherwise be a designated, but otherwise undifferentiated, portion of the suture 10. The locking portion 22 may also be a separate component that is attachable to the suture 10.

Referring to FIG. 2, a method for forming the locking portion 22 is illustrated. A portion of suture 10 may be expanded by passing a needle 11 or other rod-like wedging device through a portion of the body of the suture 10, such as through the inside of an outer braid to force the portion of the suture to expand. Once the portion of the body has been expanded, the needle 11 may be removed, leaving the portion of the body of the suture loosened and shaped to receive another portion of the suture 10, such as the first 18 and second 20 ends. Alternately, the suture may be braided over the needle 11 in the first place, and the needle 11 withdrawn to leave an expanded portion of the body.

The system 8 may be provided pre-assembled, as shown, or as separate components which may be assembled prior to, or during use. The system illustrated in FIG. 1 may be assembled by passing the stalk 14, 15 of each snare 12, 13 through the locking portion 22. Once the stalks 14,15 have been passed through the locking portion 22, the loops 16, 17 may lie on opposite sides of the locking portion 22 from their respective stalks 14, 15.

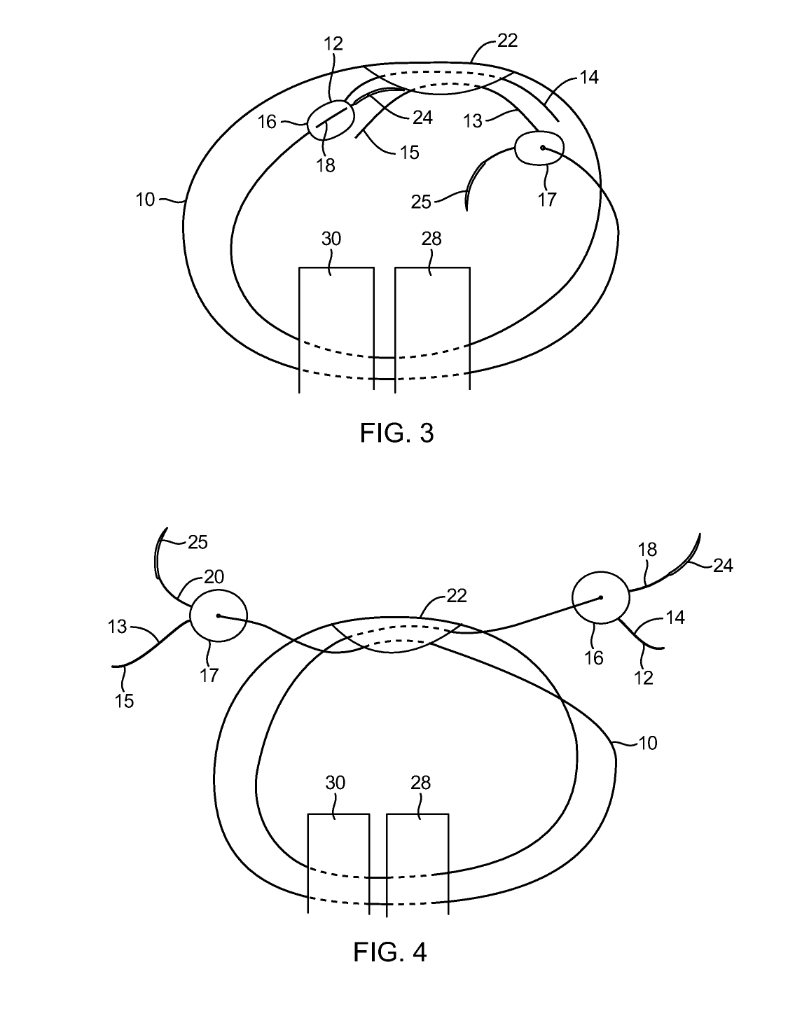

Referring to FIGS. 3-5, an example of a method of use for approximating separate portions of tissue is illustrated. The first end 18 of the suture 10 with the first needle 24 attached may be passed in a first direction through at least two portions of tissue 28, 30. The first end 18 may then be passed through the first loop 16 of the first snare 12. The second end 20 of the suture 10 with the second needle 25 attached may be passed in a second direction that is opposite the first direction through the at least two portions of tissue 28, 30, and then may be passed through the second loop 17 of the second snare 13.

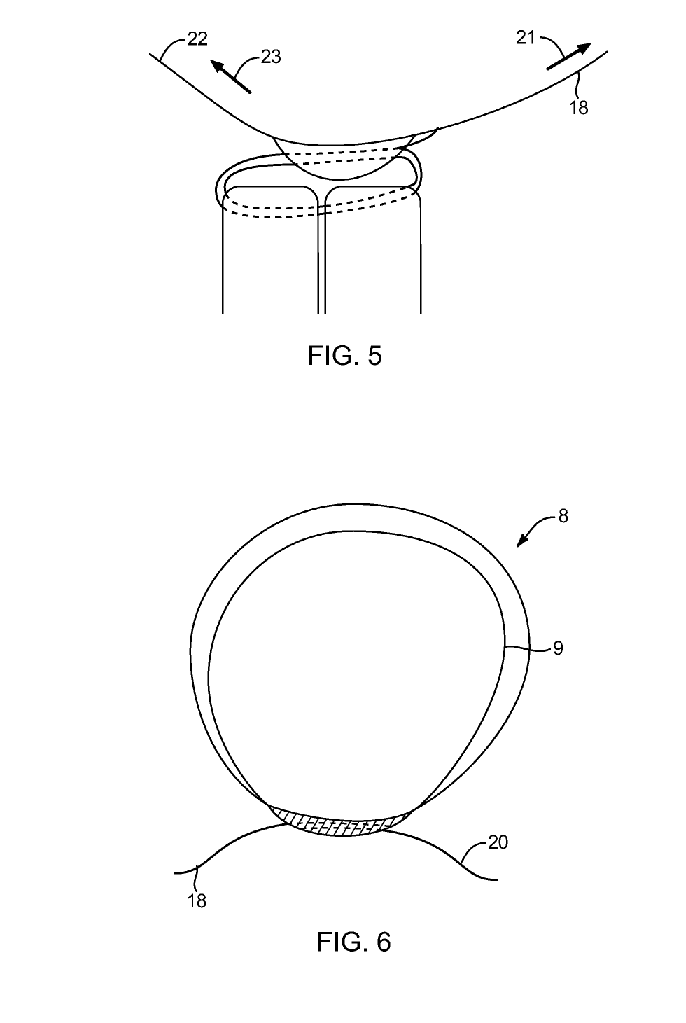

The snares 12, 13 may then be pulled simultaneously along opposite directions to draw the associated suture ends 18, 20 through the locking portion 22. In this manner, the suture 10 may be formed into a double loop. As the snare loops 16, 17 are passed through the locking portion 22, the loops 16, 17 may be compressed or flattened. Once the suture ends 18, 20 have been pulled through the locking portion 22, the ends 18, 20 may be removed from the loops 16, 17 and the snares 12, 13 may be discarded. The ends 18, 20 may then be pulled in order to tighten the double loop of suture 10 to approximate the portions of the tissue, as illustrated by motion arrows 21 and 23 in FIG. 5.

As illustrated in FIG. 6, the system 8 may be scaled up for alternative uses, such as a tourniquet. The system 8 may be provided pre-looped with the suture ends already passed through the locking portion and the snares removed. The resulting loop 9 may be passed over a portion of tissue and the suture ends may be pulled to tighten the loop around the tissue.

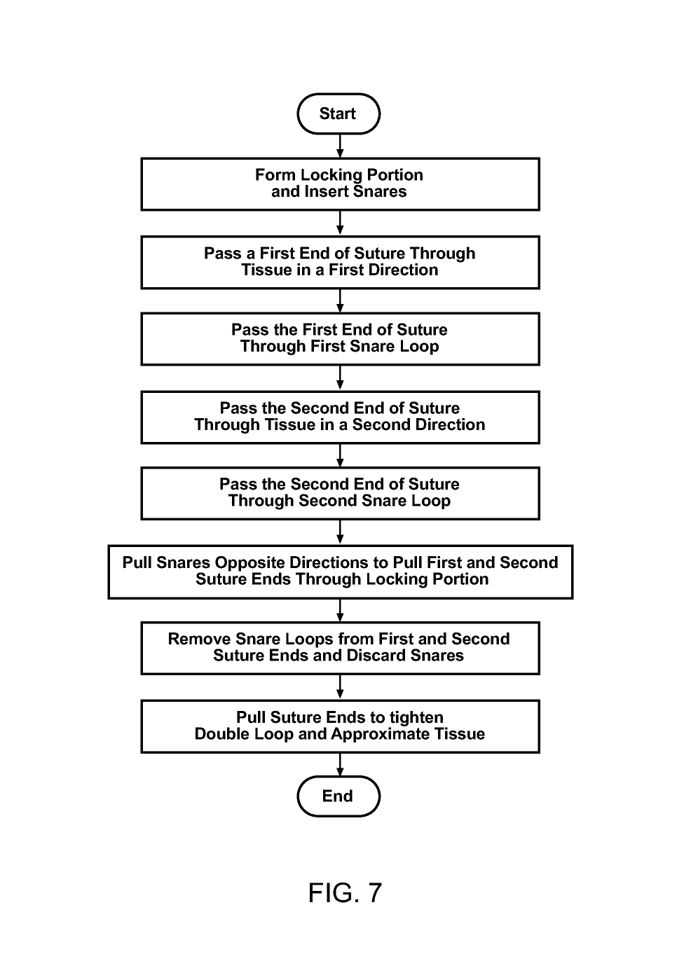

Referring to FIG. 7, a diagram illustrates a method of approximating tissue, for example, using the tissue approximation system described in FIGS. 1-6. The method starts with step 300, although it is appreciated that this method may occur after another method. In step 310, a locking portion may be created, for example, by passing a needle through a portion of a suture as previously described in FIG. 2. The locking portion may contain snares, such as in system 8. In step 320, a first end of suture may be passed through at least one tissue portion in a first direction, and then may subsequently be passed through a first snare loop, as described in step 330. A second end of suture may then be passed through at least one tissue in a second direction in step 340, and may then be passed through a second snare loop in step 350. In step 360, the snares may be pulled in opposite directions to pass the first and second suture ends through the locking portion to create a double-loop structure in the suture. In step 370, the first and second suture ends may be removed from the snare loops, and the snares may be discarded. The first and second suture ends may then be pulled to tighten the double loop around the tissue portions, pulling the tissue portions together. At this point, the method ends with step 390, although it is appreciated that another method may follow.

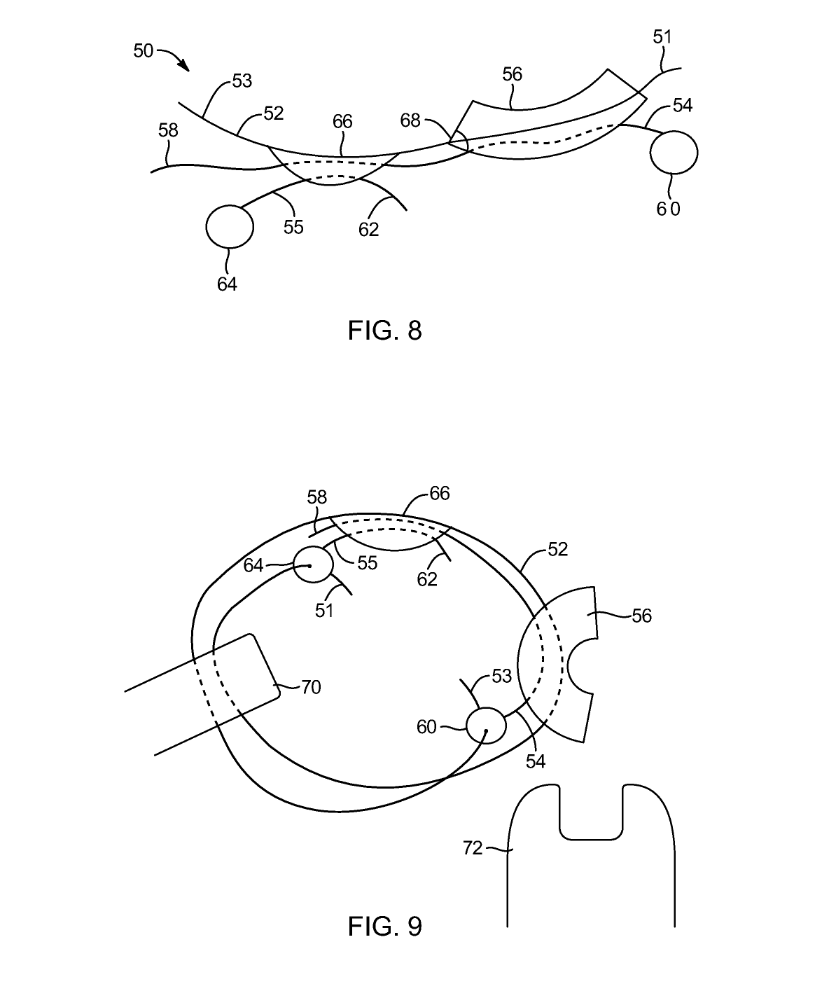

Referring to FIG. 8, another tissue approximation system 50 may include at least one strand of suture, at least one snare, at least one locking portion and at least one suture anchor. The suture and snare in this system may share some or all of the characteristics set forth above for the system of FIGS. 1-6.

The system 50 of FIG. 8 is illustrated with a single strand of suture 52, a first snare 54, a second snare 55, and a single suture anchor 56. The first snare 54 may include a first stalk 58 and a first loop 60. The second snare 55 may include a second stalk 62 and a second loop 64. The suture 52 may include a locking portion 66 similar to locking portion 22. The suture may also extend between a first end 51 and a second end 53. The suture anchor 56 may be described as a soft, somewhat tubular anchor. However, other suture anchor configurations are contemplated. Any number of suture strands, snares, or anchors may be included.

The system of FIG. 8 may be assembled in a manner that builds upon that described above for the system of FIG. 1. For example, the system 50 may be assembled by forming the locking portion 66, passing the first stalk 58 and the second stalk 62 through the locking portion 66 such that the loops 60, 64 lie on opposite sides of the locking portion. As illustrated in FIG. 8, the first snare 54 may also be passed through the anchor 56. In the example shown in FIG. 8, the first snare 54 has been passed through the anchor 56 so that the locking portion 66 and the anchor 56 are side-by-side between the loops 60, 64 of snares 54, 55. The suture anchor 56 may be connected to the first end 51 of suture 52. For example, the first end 51 may pass through a hole 68, lumen, aperture, or passageway in the anchor 56. The system 50 may be provided pre-assembled, as shown, or as separate components which may be assembled prior to, or during, use.

The system 50 may be used to approximate separate portions of tissue. For example the system 50 may be used to approximate a soft tissue to a bone.

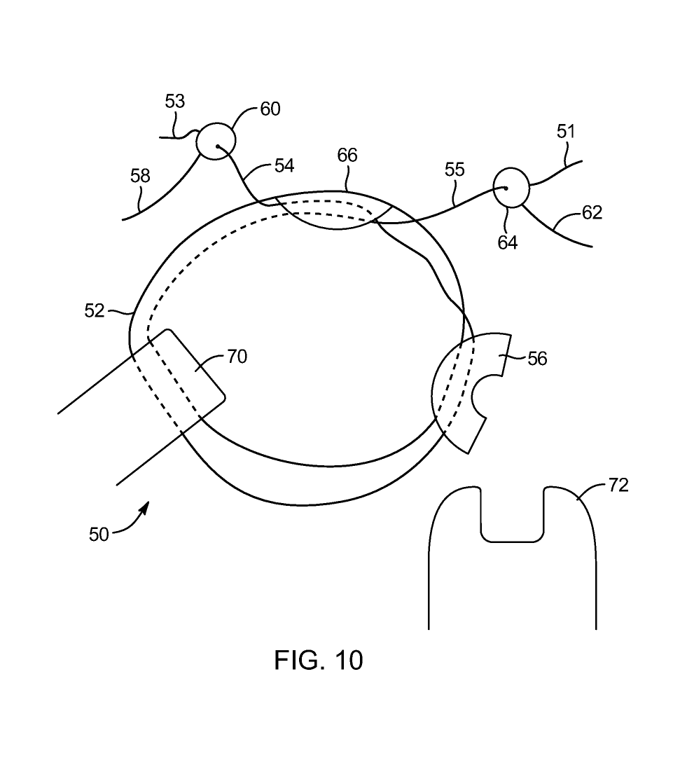

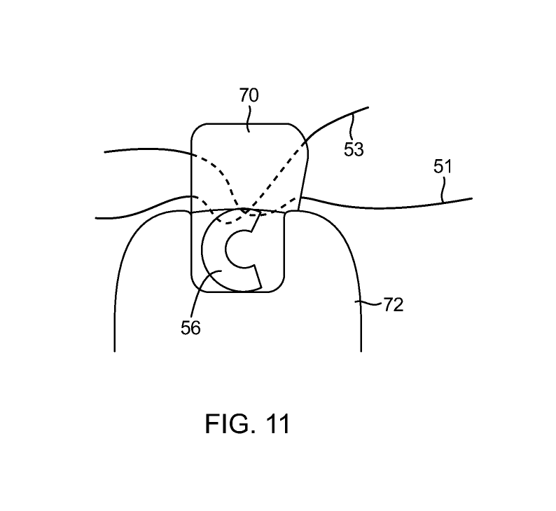

Referring to FIGS. 9-11, one example of a method of use is illustrated. The first end 51 of the suture 52 may be passed in a first direction through at least a first portion of tissue 70, and subsequently through the first loop 60 of the first snare 54. Likewise, the second end 53 of the suture 52 may be passed in a second direction opposite the first direction through the first portion of tissue 70 and subsequently through the second loop 64 of the second snare 55. The snares 54, 55 may then be pulled along opposite directions through the locking portion 66, carrying with them the first end 51 and the second end 53 of the suture 52. In this example, the first loop 60 of the first snare 54, with the first end 51 attached, travels through the locking portion 66, thus drawing the first end 51 through the locking portion. The second loop 64 of the second snare 55 with the second end 53 travels through the suture anchor 56, and then through the locking portion 56.

In this manner, the suture may be formed into a double looped structure. The loops 60, 64 of the snares 54, 55 may compress or flatten as they pass through the locking portion 66 or the anchor 56. Once the ends 51, 53 emerge from the locking portion 66, the snares 54, 55 may be removed and discarded. The anchor 56 may then be secured to a second portion of tissue 72, such as a portion of bone, tendon or ligament. This may be accomplished by deploying the anchor 56 in a hole in the bone 72, for example. The ends 51, 53 may then be pulled in order to tighten or shrink the double loop of suture to approximate the first tissue 70 to the second tissue 72.

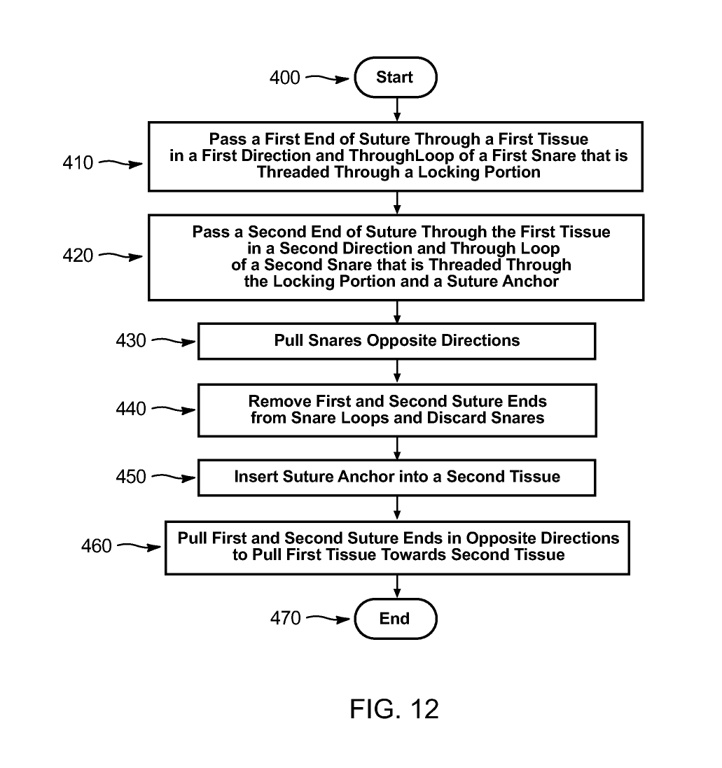

Referring to FIG. 12, another example method of approximating tissue is described, for example, using the system described in FIGS. 8-11. The method starts with step 400, although it is appreciated that this method may occur after another method, such as the formation of a locking portion and preassembly of a tissue approximation system, for example, system 50. The system may include a first snare which is threaded through a locking portion, for example, locking portion 66. The system may further include a second snare that is threaded through both the locking portion and a suture anchor, for example, suture anchor 56. In step 410, a first end of a suture may be passed through a first tissue, such as a tendon or ligament or other soft tissue in a first direction, and through the loop of the first snare. A second end of the suture may then be passed through the first tissue in a second direction and through the loop of the second snare in step 420. In step 430, the snares may be pulled in opposite directions to draw the first end of the suture through the locking portion, and to draw the second end of the suture both through the locking portion and through the suture anchor. In step 440, the first and second suture ends may be removed from the snare loops and the snares may be discarded. In step 450, the suture anchor may be inserted into a second tissue, such as a bone. In step 460, the first and second suture ends may be pulled in opposite directions to draw the first tissue towards and to contact the second tissue, thus approximating the tissue. This method may be of particular relevance for approximating a soft tissue to a bone. The method ends with step 470, although it is appreciated that this method may occur before another method, such as additional tightening or securing of the suture to the tissues using additional knots or external locking components.

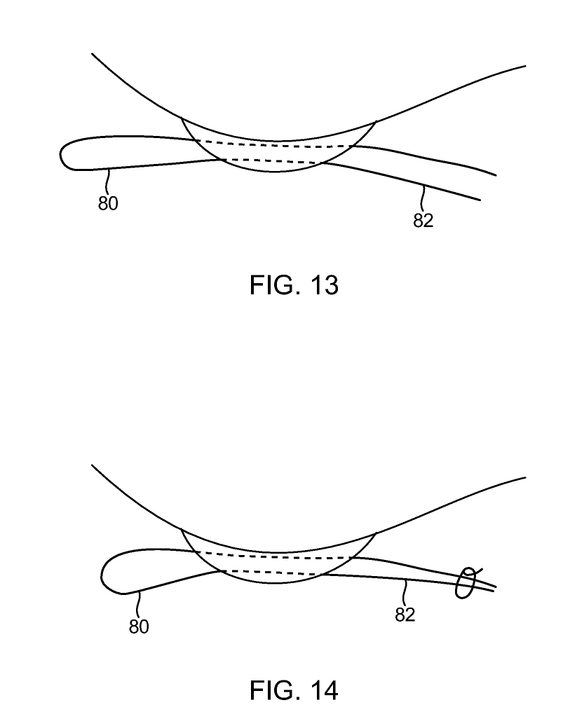

Referring to FIGS. 13 and 14, alternative examples of a snare are illustrated. Referring to FIG. 13, the snare 80 may be formed by folding a strand, such as a wire or suture, in half so that the fold or bend forms a loop. The tails may lie substantially parallel to one other to form a stalk 82. The tails may also be tied or bound together, as illustrated in FIG. 14.

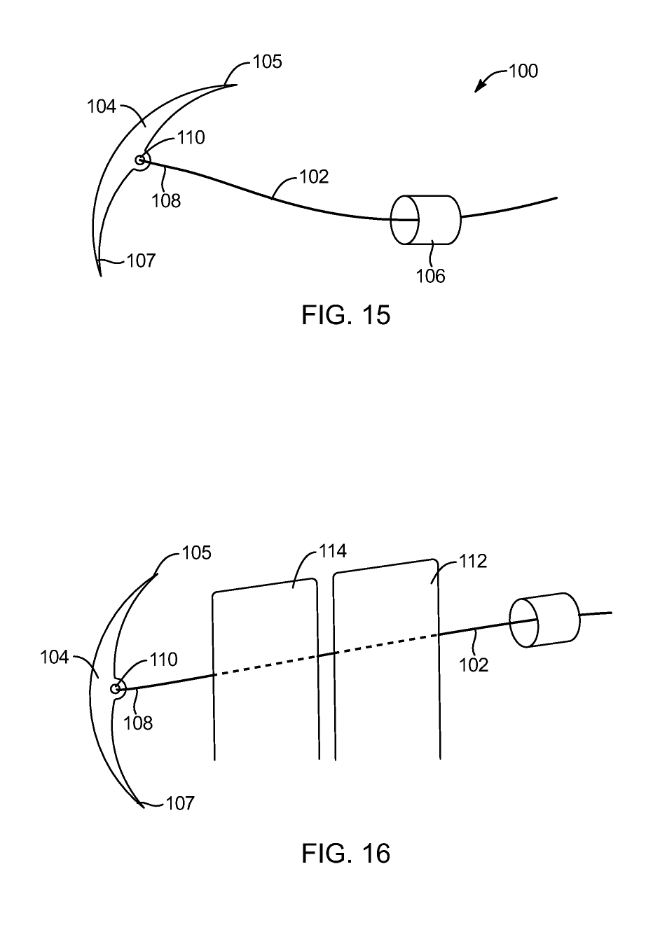

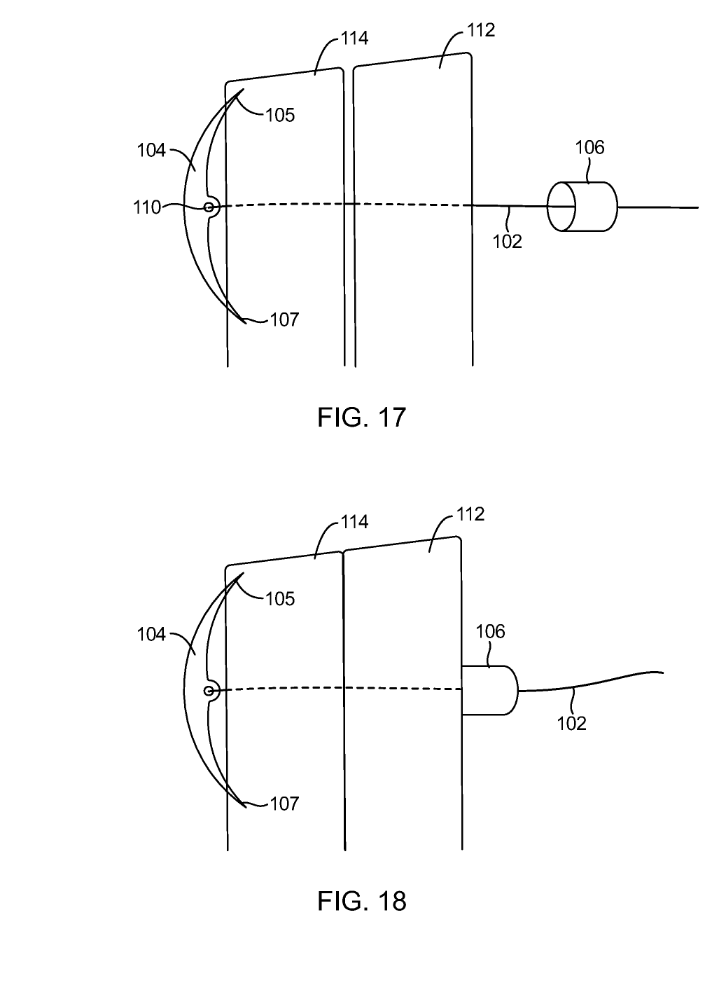

Referring to FIGS. 15-18, another example of a tissue approximation system is illustrated. System 100 includes a single suture 102, a tissue-piercing component 104 and a slidable attachment device 106. The suture 102 may be similar to suture 10, and may include at least a first end portion 108 that is attached to the tissue-piercing component 104. The tissue-piercing component 104 may also be referred to as a needle, and may include a threading-aperture 110. In the example shown in FIG. 15 the threading-aperture 110 may be located in a central portion of the needle 104. The first end portion 108 may be attached to the needle 104 at the aperture 110. The needle 104 may be arc-shaped or otherwise curved and include a first tip 105 and a second tip 107. The first and second tips 105, 107 may be pointed or roughened to dig into a tissue, such as a bone, ligament or tendon.

The slidable attachment device 106, which may also be referred to as a sliding bumper, may be carried on the suture 102, and may be slidable along the suture 102 in only one direction. The sliding bumper 106 may include a cylindrical body, or may otherwise be polygonal or irregularly shaped. The slidable bumper may include a locking portion as described above, and may function similarly. The system 100 may be provided pre-assembled, as shown, or as separate components which may be assembled prior to, or during, use.

An example of the method of use for system 100 is illustrated in FIGS. 16-18. The needle 104 and attached first end portion 108 of suture 102 may be passed through a first tissue 112 and a second tissue 114 to be approximated. Once the needle 104 has exited the second tissue 114, it may be oriented to lie substantially parallel to the surface of the second tissue 114. The suture 102 may then be pulled back in a second direction that is opposite the first direction such that the needle 104 engages a surface of the second tissue 114, and the tips 105, 107 may grip or dig into a portion of the second tissue 114. The slidable bumper 106 may then be moved along the suture in the first direction such that the slidable bumper 106 contacts a surface of the first tissue 112, to press the second tissue 114 to the first tissue 112. The remaining suture 102 that extends outward from the slidable bumper 106 may then be removed.

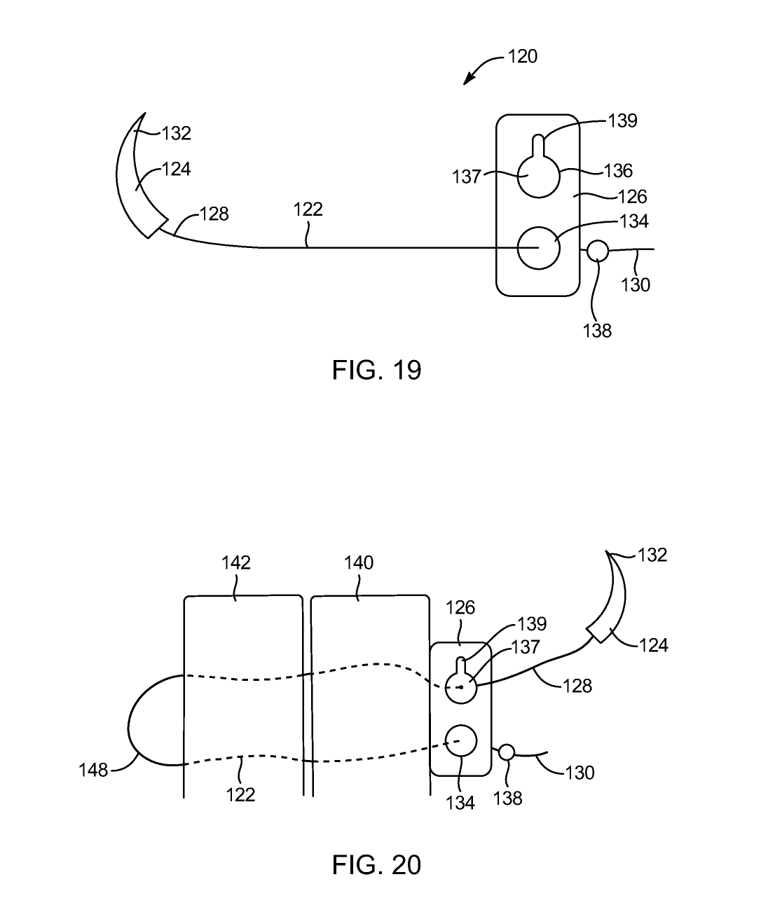

Referring to FIGS. 19-20, another example of a tissue approximation system is illustrated. System 120 may include at least a single suture 122, a needle 124 and an anchor plate 126. The single suture 122 may include a first end portion 128 and a second end portion 130. The first end portion 128 may be attachable to the needle 124. As illustrated in FIG. 20, the needle 124 may include a curved tissue piercing portion 132. The anchor plate 126 may be attached to the second end portion 130 of suture 122. The anchor plate 126 may be generally rectangular. Other geometries, such as circular, elliptical or polygonal are also contemplated. The anchor plate may include at least a first aperture 134 and a second aperture 136. The suture 122 may be attached to the anchor plate 126 by a knot 138 or other interference device that prevents pullout of the second end portion 130 from the first aperture 134. The second aperture 136 may be shaped to receive and capture or lock a portion of the suture 122 within the aperture 136. The second aperture 136 may include a rounded portion 137 and a channel portion 139.

Referring to FIG. 20, a method of use for system 120 is shown. The needle 124 attached to the first end portion 128 may be passed through a first tissue 140 and a second tissue 142 along a first direction 144. The needle 124 and attached first end portion 128 may be pulled such that the anchor plate 126 attached to the second end portion 130 engages a surface of the first tissue 140. The needle 124 and attached first end portion 128 may then be passed back through the first and second tissues 140, 142 in a different location along a second direction 146 opposite the first direction 144, forming a suture loop 148 that extends from a surface of the second tissue 142. The needle 124 and attached first end portion 128 may also be passed through the rounded portion 137 of the second aperture 136 of the anchor plate 126. The needle 124 and attached first end portion 128 may be pulled to tighten the portion of the suture 122 that was contained within the suture loop 148 against the surface of the second tissue 142, and to approximate the first tissue 140 to the second tissue 142. The first end portion 128 may also be positioned such that it becomes captured within the channel 139 of the second aperture 136, preventing backout of the suture from the plate 126. Excess suture length may be cut and removed.

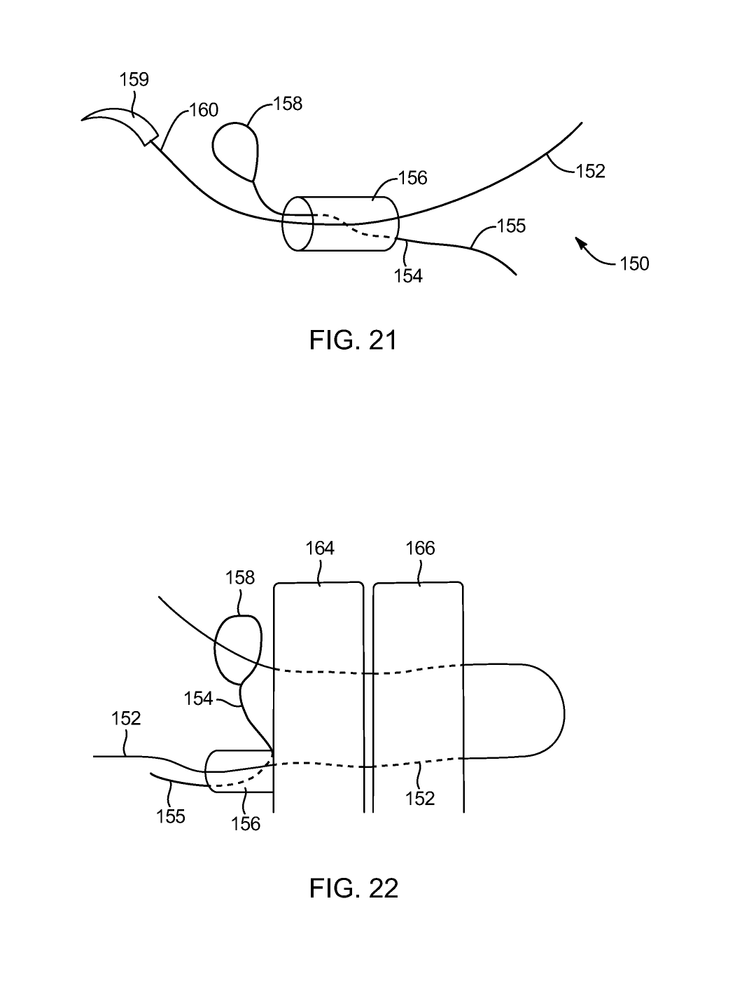

Referring to FIG. 21, another example of a system used to approximate tissue is illustrated. System 150 includes at least one flexible member 152, at least one snare 154, a tubular sleeve member 156 and a needle 159. Flexible member 152, which may also be referred to as a suture, may include a first end portion 160, which is attached to the needle 159. The needle 159 may be curved or straight.

The system 150 may be assembled such that the tubular sleeve 156 member is carried by the suture 152. The snare 154 may be similar to the snares described in previous examples, and may include a stalk 155 and a suture-capture loop 158, which may otherwise be a hook or other capture feature. The snare 154 may be passed through the tubular member 156 such that the stalk 155 and the loop 158 extend from opposite ends of the tubular member 158. The system 150 may be provided pre-assembled, as shown, or as separate components which may be assembled prior to, or during, use.

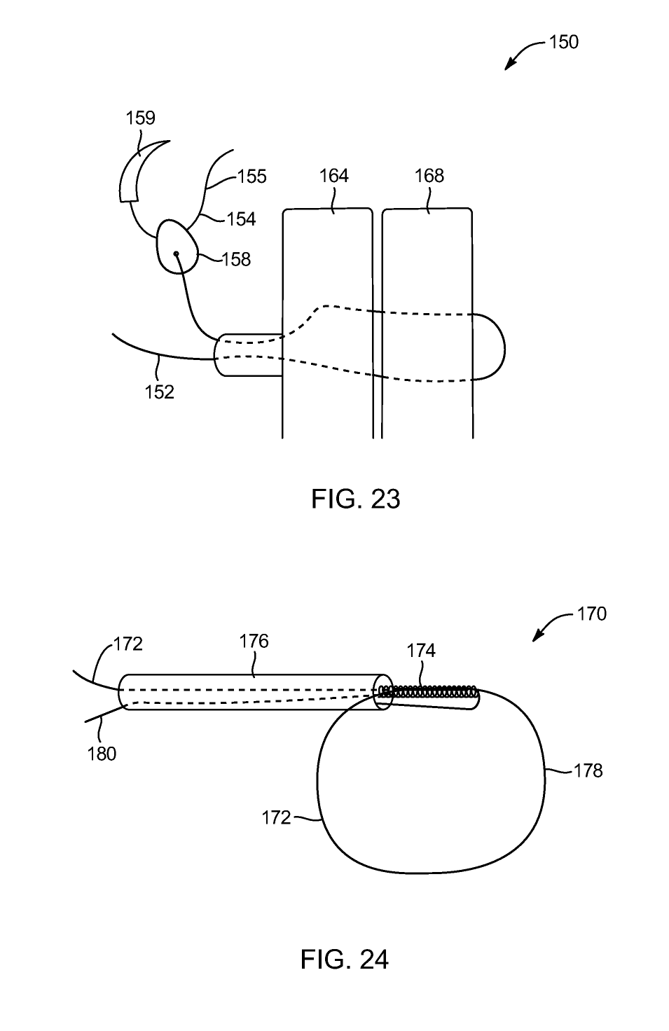

Referring to FIGS. 22-23, one example of a method of use for system 150 is illustrated. The needle 159 may be passed through a first tissue 164 and through a second tissue 166 in a first direction. When the needle 162 has been completely passed through the first 164 and second 166 tissue portions, the tubular member 156 may contact a surface of the first tissue 164. The needle 159 may then be passed back through the first and second 164, 166 tissue portions along a second pathway in a second direction that is opposite the first direction. When the needle 159 exits the first tissue 164 in the second direction, it may also be passed through the capture loop 158 of the snare 154 and pulled outward from the surface of the first tissue 164 to bring the first and second tissue 164, 166 together. When the needle 159 has been pulled through the loop 158, a portion of the suture 152 may extend through the loop 158. The snare 154 may then be retracted to tighten and secure the tubular sleeve 156 against the first tissue 164 and fix the tubular sleeve 156 on the suture 152. Excess suture may be cut and removed.

Referring to FIG. 24, another example of a system for tissue approximation is described. System 170 includes a flexible member 172, which may also be referred to as a suture. The suture 172 may include a locking portion 174, which may be formed similar to locking portion 22. The locking portion 174 may be anchored to or attached to an end of an elongated tubular shaft 176. The suture 172 may be formed into a loop structure 178. The loop structure 178 may be pre-formed. When the suture 172 is formed into the loop structure 178, at least one end of the suture 172 may have been passed through the locking portion 174 in a first direction. A second end 180 of the suture 172 may be passed through the locking portion 174 in along a direction opposite to the first direction, and then passed through the elongated tubular shaft 176. The suture 172 may be slidable within the elongated tubular shaft 176.

The preformed loop 178 may be used to grab a piece of tissue or several pieces of tissue by placing the loop 178 around the tissue. The second end 180 may then be pulled through the elongated tubular shaft 176 to tighten the loop 178 around the tissues to approximate the tissues.

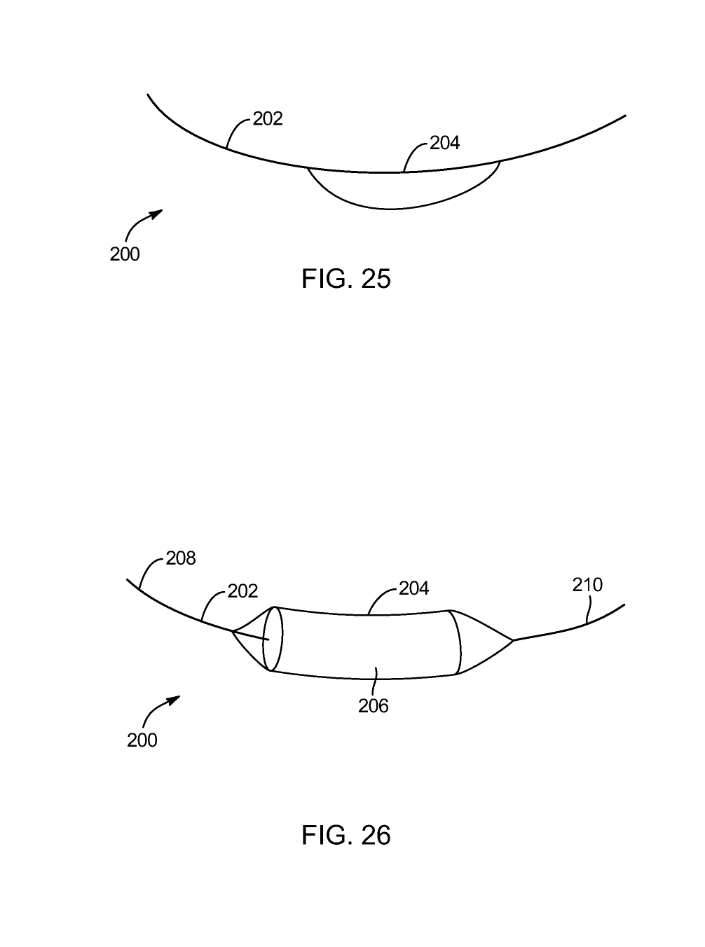

Referring to FIG. 25, another example of a system for tissue approximation is described. System 200 may include a flexible member 202, which may also be referred to as a suture. The flexible member 202 may include a locking portion 204 that is formed similar to locking portion 22. Once a portion of the suture 202 has been expanded to create a locking portion 204, a tubular member 206 may be inserted into the expanded locking portion 204 to temporarily hold the lock portion 204 open, as illustrated in FIG. 26. The tubular member 206 may be cylindrical, and may include a seam that allows the body of the tubular member 206 to be opened along the seam. Other configurations, such as polygonal or partial cylinders are also contemplated.

The suture 202 may include a first end portion 208 and a second end portion 210. A needle or other tissue-piercing device may be attached to at least one of the end portions 208, 210. The system 200 may be applied to approximate tissue in a method that is similar to the method described for system 8. The first and second end portions 208, 210 may be first be passed in opposite directions through a first and second tissue. The first and second ends 208, 210 may then be passed through the tubular member 206. The tubular member 206 may then be removed by opening the tubular member 206 along the seam, and the suture 202 may be cinched down on itself by pulling the first and second ends 208, 210 through the locking portion 204.

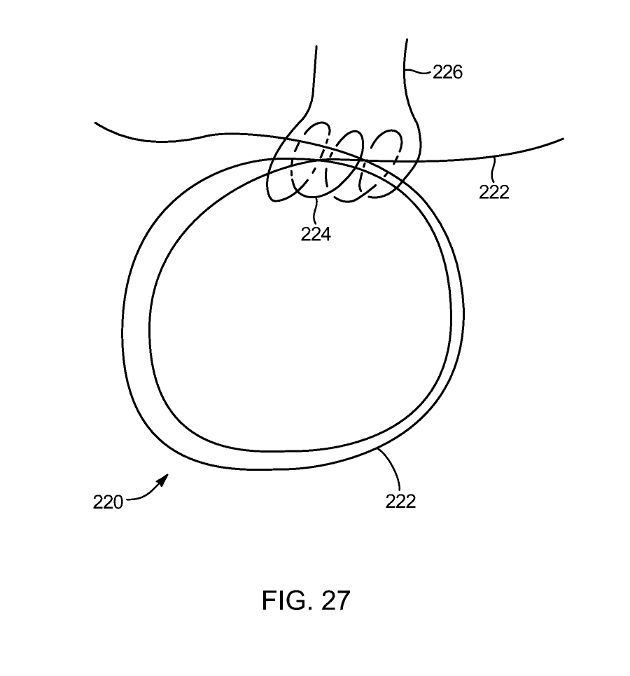

Referring to FIG. 27, another system for tissue approximation is illustrated. System 220 is similar to system 8 described in FIGS. 1-6. System 220 may include a first flexible member 222, such as a suture, and a locking portion 224 that is formed using a second flexible member 226. The second flexible member 226 may also be suture, and may be separate from the first flexible member 226. The second flexible member 226 may also be made of a different material, such as wire or string. The second flexible member 226 may be wrapped around the first flexible member 222 to form the locking portion 224. The locking portion 224 may be formed with a pre-tied knot that only requires pulling a first and second end of flexible member 222 through the locking portion 224 to cinch the first suture 222 down on itself. The suture 222 may have been passed through or around a first and second tissue portion to approximate the tissues when the suture 222 is cinched.

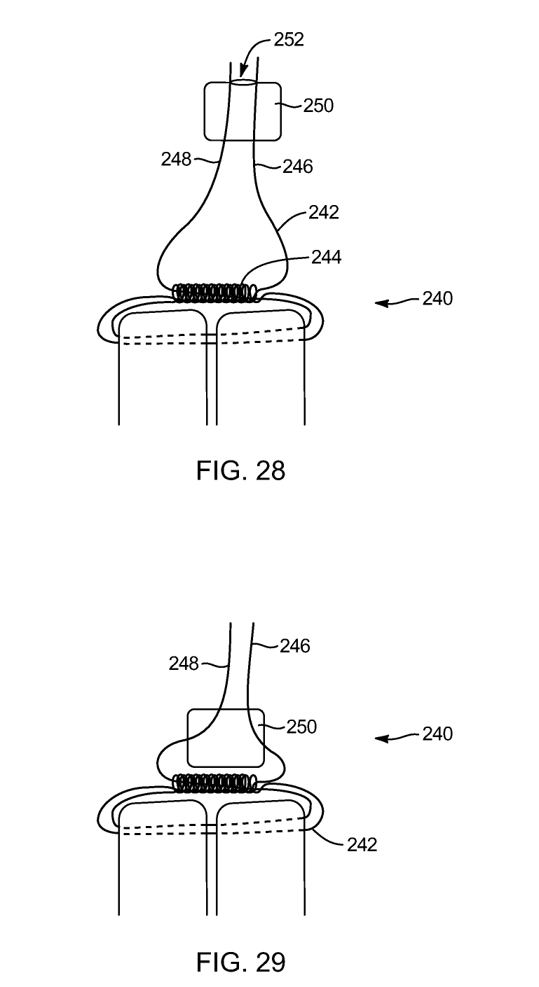

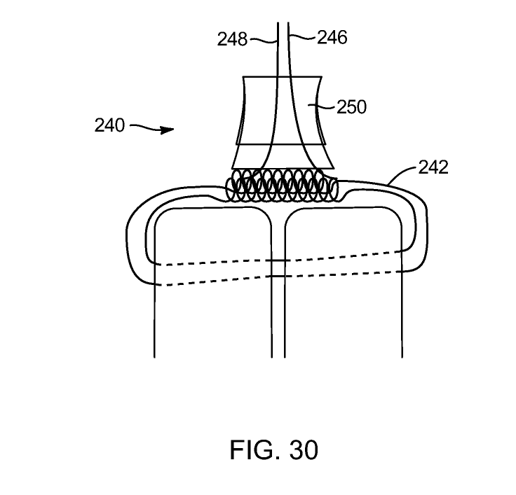

Referring to FIGS. 28-30, another system for approximating tissue is illustrated. System 240 may be similar to system 8, and may include a first suture 242, and a locking portion 244. The first suture may include a first end 246 and a second end 248. The system 240 may be positioned within a first tissue and a second tissue in a manner that is similar or identical to system 8. Once the first end 246 and the second end 248 have been passed through the tissue portions and through the locking portions, the ends 246, 248 may be passed through a secondary lock tube 250. The lock tube 250 may include a central aperture 252, through which both ends are passed. The lock tube 250 may be slidable along the suture 242, and may be slid until the lock tube 250 contacts the locking portion 244. The lock tube 250 may then be securably attached to the locking portion 244 via crimping as illustrated in FIG. 30, or by another means, to secure the two tissue portions together.

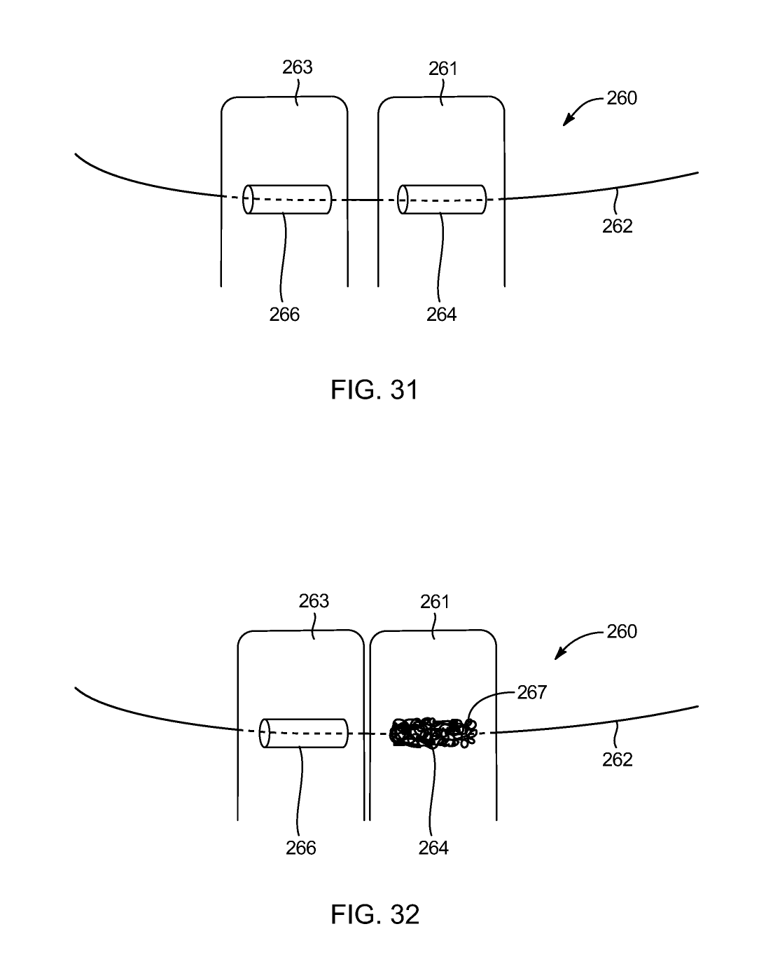

Referring to FIGS. 31 and 32, another example of a system for tissue approximation is illustrated. System 260 may include a flexible member 262 that may be threaded through a first tissue 261 and a second tissue 263.

The flexible member 262 may include a first exterior sheath 264 and a second exterior sheath 266. The flexible member 262 may be generally cylindrical. The first and second exterior sheaths 264, 266 which may also be referred to as exterior windings, may be carried on the flexible member 262 and spaced apart from one another. The exterior sheath 266 may be slidable along the length of the core component 264.

After the flexible member 262 has been threaded through the first and second tissue portions 261, 263, the flexible member 262 may be positioned such that the first sheath 264 lies within the first tissue portion 261 and the second sheath 266 lies within the second tissue portion 263. The flexible member 262 may be pulled along a first direction 265 away from the surface of the second tissue portion 263, as illustrated in FIG. 32. When the flexible member 262 is pulled along the first direction 265, the first exterior sheath 264 may become bunched or otherwise compressed to expand radially outward from the flexible member 262 as illustrated in FIG. 32. Upon expansion outward, a bunched portion 267 may engage the first tissue portion 261 to draw the first tissue portion 261 into contact with the second tissue portion 263 to approximate the two tissue portions 261, 263. The system may be secured by tying a knot or by using an external locking mechanism, for example, tubular sleeve 156.

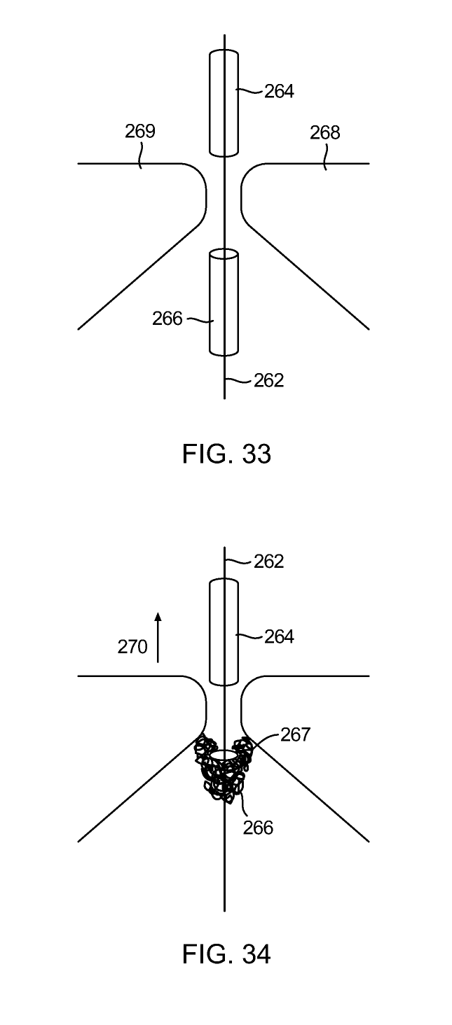

Referring to FIGS. 33 and 34, another example of a method of use for flexible member 262 is contemplated. In use, a portion of the flexible member 262 may be used to anchor the flexible member 262 between two pieces of tissue, for example between two pieces of bone 268, 269. A portion of flexible member 262 containing the second sheath member 266 may be positioned between the two pieces of bone 268, 269. Another portion of flexible member 262 containing first sheath member 264 may then be pulled in a first direction 270. When the flexible member 262 is pulled in the first direction 270, the exterior sheath 266 may become bunched, creating a bunched section 267 or otherwise compressed to expand radially outward from the core component 264 as illustrated in FIG. 34. Upon expansion outward from the core component 264, the exterior sheath may engage the two pieces of bone 268, 269 and prevent the pullout of the flexible member 262.

The present disclosure may be embodied in other specific forms without departing from its spirit or essential characteristics. It is appreciated that various features of the above-described examples can be mixed and matched to form a variety of other alternatives. As such, the described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

1. A system for approximating tissue, comprising:
a first suture carrier feature; and
a flexible member, wherein the flexible member is temporarily coupled to the first suture carrier feature, wherein the flexible member comprises a first end portion and a second end portion, wherein the flexible member comprises a lock portion between the first end portion and the second end portion, wherein the lock portion is integral to the flexible member.

2. The system of claim 1, wherein the flexible member comprises a body, wherein the lock portion comprises an expanded portion of the flexible body.

3. The system of claim 1, wherein the first suture carrier comprises a snare, wherein the snare comprises at least a partial loop and a stalk, wherein when the first suture carrier is temporarily coupled to the flexible member, a portion of the stalk is contained within the lock portion.

4. The system of claim 1, wherein the system comprises a second suture carrier feature, wherein the first suture carrier feature is attachable to the first end portion and the second suture carrier portion is attachable to the second end portion.

5. The system of claim 4, wherein the flexible member is temporarily coupled to the second suture carrier feature.

6. The system of claim 4, wherein when the first suture carrier is attached to the first end portion, the first suture carrier and first end portion are slidable within the lock portion.

7. The system of claim 1, wherein the first end portion comprises a tissue piercing component.

8. A system for approximating tissue, comprising:
at least one suture carrier feature; and
a flexible member, wherein the flexible member is temporarily coupled to the suture carrier feature, wherein the flexible member comprises a first end portion and a second end portion, wherein the flexible member comprises a lock portion between the first end portion and the second end portion, wherein the lock portion is integral to the flexible member; and
a suture anchor, wherein the suture anchor is carried by the flexible member.

9. The system of claim 8, wherein the flexible member comprises a body, wherein the lock portion comprises an expanded portion of the body.

10. The system of claim 8, wherein the at least one suture carrier comprises a first snare and a second snare, wherein the first snare comprises a first at least partial loop and a first stalk, wherein when the first snare is temporarily coupled to the flexible member, a portion of the first stalk is contained within the lock portion, wherein the second snare comprises a second at least partial loop and a second stalk, wherein when the second snare is temporarily coupled to the flexible member, a portion of the second stalk is contained within the lock portion and the suture anchor.

11. The system of claim 8, wherein the suture anchor is shaped to be inserted into a tissue.

12. The system of claim 8, wherein the suture anchor is between the lock portion and the second end portion.

13. The system of claim 8, wherein the second end portion is slidable within the lock portion.

14. The system of claim 8, wherein the first end portion is slidable within the lock portion and the suture anchor.

15. A method of tissue approximation, comprising:
providing a flexible member, wherein the flexible member comprises a first end portion, a second end portion, and a lock portion, wherein the lock portion is between the first and second end portions;
providing a snare, wherein the snare comprises a loop and a stalk, wherein the loop is shaped to receive a portion of the flexible member, wherein the stalk is slidably received by the lock portion;
sliding the stalk into the lock portion such that the loop is adjacent the first end portion;
passing the second end portion through at least one tissue and subsequently through the loop;
sliding the stalk through the lock portion to draw the second end portion through the lock portion; and
drawing at least a portion of the strand through the lock portion.

16. The method of claim 15, wherein the flexible member comprises a suture anchor, wherein the suture anchor is located between the lock portion and the second end portion.

17. The method of claim 16, wherein the stalk is slidably received by the lock portion and the suture anchor.

18. The method of claim 17, wherein the second end portion is passed through at least one tissue and subsequently through the loop, wherein the stalk is slid through the lock portion and the suture anchor to draw the second end portion through the lock portion and suture anchor.

19. The method of claim 15, wherein the flexible member comprises a body, wherein the lock portion comprises an expanded portion of the flexible body.

20. The method of claim 18, wherein the suture anchor is placed into a bone, wherein the first and second end portions are pulled in opposite directions to approximate the at least one tissue and the bone.
